(19) United States
(12) Patent Application Publication
(10) Pub. No.: US 2014/0093045 A1
(43) Pub. Date: Apr. 3, 2014

(54) PORTABLE X-RAY DIAGNOSTIC APPARATUS

(71) Applicant: CANON KABUSHIKI KAISHA, Tokyo (JP)

(72) Inventors: Tetsuo Shimada, Hachioji-shi (JP); Shinji Nishimura, Yokohama-shi (JP)

(21) Appl. No.: 14/036,156

(22) Filed: Sep. 25, 2013

(30) Foreign Application Priority Data
Sep. 28, 2012 (JP) 2012-218461

(51) Int. Cl. A61B 6/00

(52) U.S. Cl. CPC A61B 6/4405
USPC 378/98

(57) ABSTRACT

There is provided a portable X-ray diagnostic apparatus. The X-ray diagnostic apparatus comprises an X-ray generation unit configured to generate X-rays, a display unit configured to display information about X-ray imaging by the X-ray generation unit, a column including a shaft extending in a first direction and configured to be rotatable about the shaft, a cart on which the display unit and the column are set, and an extendable boom configured to support the X-ray generation unit and extend in a second direction crossing the first direction, the boom obtaining, when storing the X-ray generation unit, a length to store the X-ray generation unit between the display unit and the column.

### BACKGROUND OF THE INVENTION

1. Field of the Invention

The present invention relates to a portable X-ray diagnostic apparatus.

2. Description of the Related Art

A portable X-ray diagnostic apparatus has a function for moving to a place where a patient exists to perform X-ray imaging for the patient who is difficult to move from a hospital room because of the condition or the like or who is under a surgical operation or first-aid treatments.

FIG. 1 schematically shows the arrangement of a conventional portable X-ray diagnostic apparatus. The portable X-ray diagnostic apparatus includes an X-ray generation unit 101 including an X-ray tube that emits X-rays, and a support that supports the X-ray generation unit 101, as disclosed in, for example, Japanese Patent Nos. 4612832 and 4515921. The support includes, for example, a column 103 that is set vertically on a cart 105 of the portable X-ray diagnostic apparatus, and an extendable boom 102 that is set on the column to be movable in the vertical direction.

The portable X-ray diagnostic apparatus also includes a battery and a main body with a computer for controlling an X-ray detection unit in, for example, the cart 105. A monitor 104 of the computer is provided, above the main body. The monitor 104 includes an operation unit using, for example, a touch sensor. The computer performs, for example, management of imaging conditions, adjustment of the density and the like of captured images, and management of at least one of a list of patients scheduled for imaging and information of body parts to be captured. The portable X-ray diagnostic apparatus has the above-described column 103, for example, on the front side of the main body viewed from the operator.

The portable X-ray diagnostic apparatus is arranged near the imaging target patient. The X-ray generation unit 101 is arranged such that the X-ray tube is arranged at a position corresponding to a part to be captured by rotating the extendable boom 102 about the column 103 to adjust its direction and adjusting the distance from the column 103 by the extendable boom 102. FIG. 2 shows this state. FIG. 2 schematically shows a state in which X-ray imaging is done for a patient 202 lying on a bed 201 in a hospital room 200. An operator 203 adjusts the degree of expansion/contraction of the extendable boom 102 and the rotation amount of the column 103 to arrange the X-ray generation unit 101 at a position corresponding to the part to be captured, and performs imaging. At this time, for example, to control the X-ray detection unit, an instruction is given to the computer via the monitor 104. In this way, X-ray imaging in the hospital room 200 or the like can be conducted using the portable X-ray diagnostic apparatus. Note that when imaging is not performed, the extendable boom 102 is folded, and the column 103 is rotated to arrange the X-ray generation unit 101 at a predetermined storage location on a side of the main body, as shown in FIG. 1, thereby easily carrying the portable X-ray diagnostic apparatus.

When imaging is not performed, the X-ray generation unit 101 is arranged on the side of the main body with respect to the column, as shown in FIG. 1. In this case, the X-ray generation unit 101 is arranged above the monitor 104, and this may restrict the referable range and inputtable range of the monitor 104 for the operator at the time of storage. Hence, when the operator wants to refer to the list of patients scheduled for imaging or the like, the X-ray generation unit 101 is moved from the predetermined storage location even at the time of moving without X-ray imaging, resulting in poor operability.

The present invention provides a portable X-ray diagnostic apparatus that allows an operator to do operations even when imaging is not performed.

### SUMMARY OF THE INVENTION

According to one aspect of the present invention, there is provided a portable X-ray diagnostic apparatus comprising: an X-ray generation unit configured to generate X-rays; a display unit configured to display information about X-ray imaging by the X-ray generation unit; a column including a shaft extending in a first direction and configured to be rotatable about the shaft; a cart on which the display unit and the column are set; and an extendable boom configured to support the X-ray generation unit and extend in a second direction crossing the first direction, the boom obtaining, when storing the X-ray generation unit, a length to store the X-ray generation unit between the display unit and the column.

According to one aspect of the present invention, there is provided a portable X-ray diagnostic apparatus comprising an X-ray generation unit configured to generate X-rays, a support mechanism configured to support the X-ray generation unit and move the X-ray generation unit to a predetermined position, and an operation display unit configured to display information about X-ray imaging and accept an operation, wherein a storage location of the X-ray generation unit when the X-ray imaging is not performed is decided such that the storage location and a position of the operation display unit are spaced apart in at least one of a horizontal direction and a vertical, direction by not less than a predetermined distance.

According to one aspect of the present invention, there is provided a portable X-ray diagnostic apparatus comprising an X-ray generation unit configured to generate X-rays, a support mechanism configured to support the X-ray generation unit and move the X-ray generation unit to a predetermined position, and an operation display unit configured to display information about X-ray imaging and accept an operation, wherein the X-ray generation unit when the X-ray imaging is not performed is stored on a rear surface of a surface of the operation display unit to perform display and an operation.

Further features of the present invention will be apparent from the following description of exemplary embodiments with reference to the attached drawings.

### BRIEF DESCRIPTION OF THE DRAWINGS

FIG. 1 is a view schematically showing a conventional portable X-ray diagnostic apparatus;

FIG. 2 is a view schematically showing X-ray imaging using the conventional portable X-ray diagnostic apparatus;

FIG. 3 is a view schematically showing a portable X-ray diagnostic apparatus that stores an X-ray generation unit horizontally spaced apart from a monitor by a predetermined distance or more;

FIG. 4 is a view schematically showing a portable X-ray diagnostic apparatus that stores an X-ray generation unit vertically spaced apart from a monitor by a predetermined distance or more;

FIG. 5 is a view schematically showing the portable X-ray diagnostic apparatus including a four-stage extendable boom;

FIG. 6 is a view schematically showing a portable X-ray diagnostic apparatus that includes a monitor capable of changing the set angle and stores an X-ray generation unit on the rear surface of the monitor;

FIG. 7 is a view schematically showing the portable X-ray diagnostic apparatus that includes the monitor set above the storage location of the X-ray generation unit and stores the X-ray generation unit under the monitor;

FIG. 8 is a view schematically showing a portable X-ray diagnostic apparatus that stores an X-ray generation unit under an extendable boom;

FIG. 9 is a view schematically showing the portable. X-ray diagnostic apparatus that stores the X-ray generation unit on the extendable boom;

FIG. 10 is a side view of a multistage extendable boom including a handle set on the lower side of the second stage;

FIG. 11 is a sectional view of the multistage extendable boom including the handle set on the lower side of the second stage;

FIG. 12 is a side view of a multistage extendable boom including a handle set on the upper side the second stage;

FIG. 13 is a sectional view of the multistage extendable boom including the handle set on the upper side the second stage;

FIG. 14 is a view showing a state in which an X-ray generation unit is placed at a storage location formed as a concave portion in an X-ray diagnostic apparatus including an extendable column;

FIG. 15 is a view showing a state in which the X-ray generation unit is placed at the storage location and partially covered by side walls in the X-ray diagnostic apparatus including the extendable column;

FIGS. 16A and 16B are views showing an X-ray diagnostic apparatus including an extendable column in which the monitor is set to be slidable in a state in which the X-ray generation unit placed at the storage location is partially covered by side walls;

FIGS. 17A and 17B are views showing an X-ray diagnostic apparatus including an extendable column in which the monitor is set to be slidable in a state in which the X-ray generation unit placed at the storage location is mostly covered by side walls;

FIG. 18 is a view showing a state in which the column and the extendable boom are expanded in an X-ray diagnostic apparatus including an extendable column; and

FIGS. 19A to 19C are views showing an example of a user interface displayed on the monitor.

### DESCRIPTION OF THE EMBODIMENTS

Exemplary embodiments of the present invention will now be described in detail with reference to the drawings. It should be noted that there relative arrangement of the components, the numerical expressions and numerical, values set forth in these embodiments do not limit the scope of the present invention unless it is specifically stated otherwise.

### First Embodiment

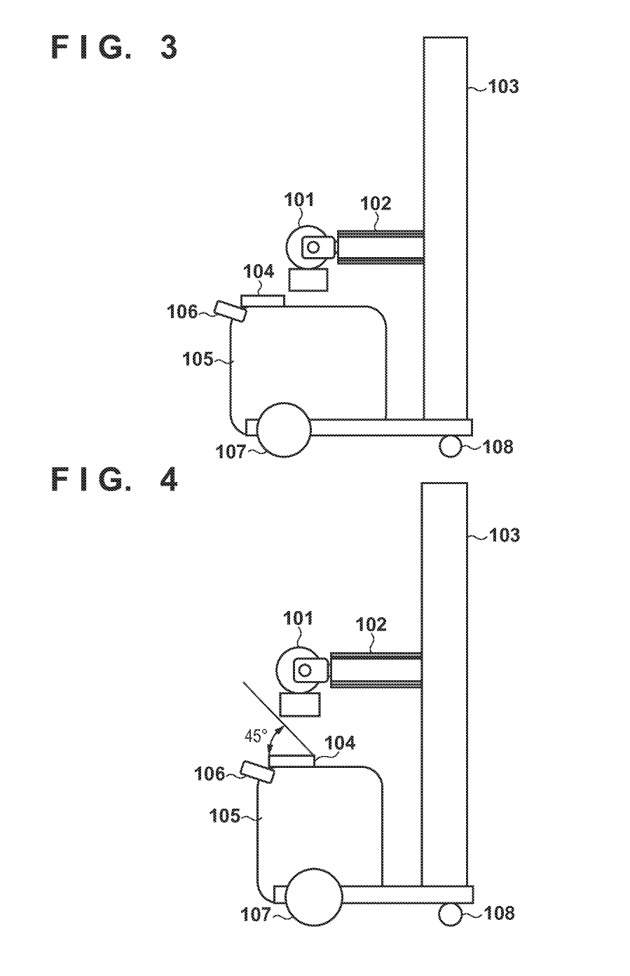

FIG. 3 is a view schematically showing the arrangement of a portable X-ray diagnostic apparatus according to this embodiment. The portable X-ray diagnostic apparatus according to this embodiment includes an X-ray generation unit 101, an extendable boom 102, a column 103, a monitor 104, and a cart 105, like the portable X-ray diagnostic apparatus shown in FIG. 1. The cart 105 is provided with two wheels on each side, that is, four wheels 107 and 108 with which the portable X-ray diagnostic apparatus can move in hospital facilities. Note that the front wheels 108 or the rear wheels 107 may include an electric motor to be driven in accordance with, for example, the operator's operation on an operation unit provided on a handle 106 and assist the portable X-ray diagnostic apparatus in moving. The cart 105 incorporates, for example, a battery and supplies power to apparatus movement, X-ray exposure, a digital plane detector (X-ray detection unit), a computer for controlling the detector, and the monitor 104. Note that the monitor 104 is an operation display unit having not only an information display function but also an operation function of accepting an operation of the computer or the like.

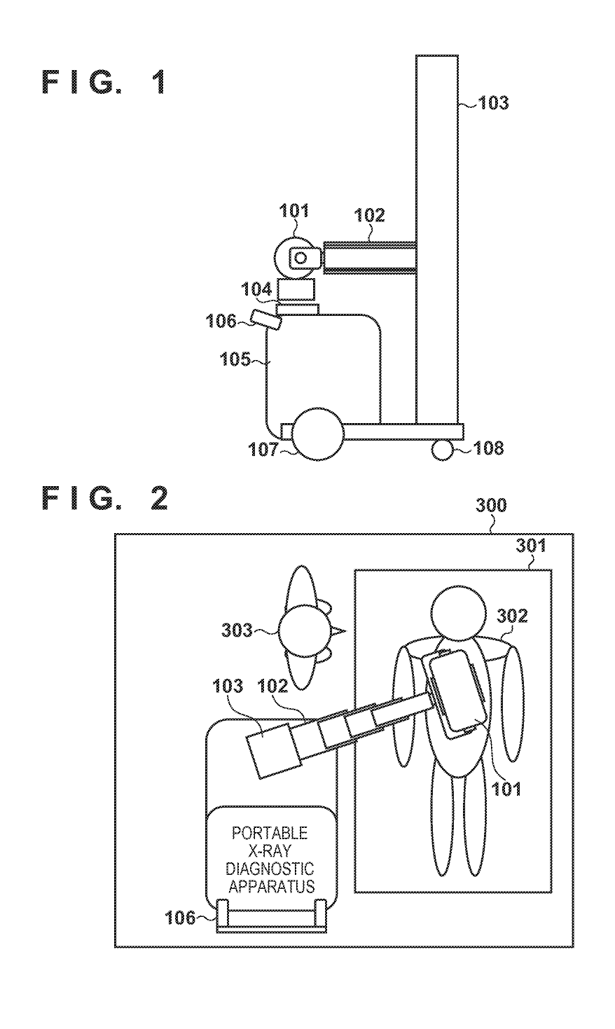

The portable X-ray diagnostic apparatus includes the column 103 extending in a first direction on the front side of the cart 105, and the extendable boom 102 joined to the column to expand or contract in a second direction (vertical direction) crossing the first direction, as shown in the example of FIGS. 1 and 2. The portable X-ray diagnostic apparatus also includes the X-ray generation unit 101 attached to the end of the extendable boom 102. The X-ray generation unit 101 is a unit including an X-ray tube. The extendable boom 102 and the column 103 constitute a support mechanism for supporting the X-ray generation unit 101 and also a moving mechanism for moving the X-ray generation unit 101 to a predetermined position. At this time of X-ray imaging, the X-ray generation unit 101 is moved to a position suitable for X-ray imaging within the movable ranges of the column 103 and the extendable boom 102. The X-ray detection unit is set and held on the extension of the X-ray focus and the imaging target body part. An X-ray tube voltage, a tube current, an imaging time, and the like optimum for the patient's imaging target body part are set, and imaging is executed. The X-ray detection unit reads an X-ray signal in synchronism with the X-ray exposure and transfers the signal to the computer. Inc computer performs image processing based on the transferred signal and converts the signal into an image effective for diagnosis. The converted image signal is sent to an image server installed in the hospital by wired connection or wireless connection and saved or browsed for diagnosis. When X-ray imaging has ended, the X-ray generation unit 101 is stored in a predetermined storage location, for example, on the rear side of the column 103 to facilitate movement. Note that the storage location is the position of the X-ray generation unit 101 when, for example, the extendable boom 102 is contracted to a predetermined length (for example, the minimum length) and arranged in a direction reverse to the traveling direction of the portable X-ray diagnostic apparatus, that is, behind the column in the traveling direction. Even when the portable X-ray diagnostic apparatus comes into contact with an obstacle on the front side, the X-ray generation unit 101 is protected. In addition, she portable X-ray diagnostic apparatus becomes sufficiently compact when moving. For these reasons, the portable X-ray diagnostic apparatus can easily be moved.

In the portable X-ray diagnostic apparatus according to this embodiment, when X-ray imaging is not executed at this time of, for example, movement, the storage location of the X-ray generation unit 101 does not impede the operator's operation on the monitor 104, as shown in FIG. 3. That is, she storage location of the X-ray generation unit 101 is predetermined as a position where the stored X-ray generation unit 101 does not cover the operation display unit for information display and operations of the portable X-ray diagnostic apparatus. That is, the length of the extendable boom 102, the rotation amount of the column 103, and the position of the monitor 104 at the storage location are determined so that the contents of information displayed on the monitor 104 at the storage location of the X-ray generation unit 101 are visible. In the example shown in FIG. 3, the horizontal position of the stored X-ray generation unit 101 and the horizontal position of the monitor 104 are spaced apart by a predetermined distance or more. In this case, when the X-ray generation unit 101 is stored, the respective units are arranged such that the X-ray generation unit 101 is arranged on the front side of the monitor 104, and the column is arranged on the front side of the X-ray generation unit 101 in the traveling direction of the portable X-ray diagnostic apparatus. Since the monitor 104 is arranged at the hindmost position in the traveling direction, the operation is easy. This arrangement facilitates the operation especially when the monitor 104 is a touch panel input device.

Note that the predetermined distance here may be decided proportional to, for example, the sizes of the monitor 104 and the X-ray generation unit 101. For example, the predetermined distance may be decided such that the distance between the center of the monitor 104 on the horizontal plane and the center of the X-ray generation unit 101 on the horizontal plane becomes equal to or more than ½ the total length of the monitor 104 and the X-ray generation unit 101 along the anteropostior axis of the portable X-ray diagnostic apparatus. Any other distance can be decided as the predetermined distance as long as information browsing or a predetermined operation can be done on the monitor 104 when the X-ray generation unit 101 is stored.

The operator who operates the portable X-ray diagnostic apparatus powers on the portable X-ray diagnostic apparatus upon receiving an X-ray imaging instruction in a ward, transfers the information of the imaging target patient to the computer for controlling the X-ray detection unit, and moves the portable X-ray diagnostic apparatus to the hospital room while referring to the contents. In the portable X-ray diagnostic apparatus according to this embodiment, since the monitor 104 is not wholly covered by the X-ray generation unit 101, the operator can perform an operation to refer to or input information via the monitor 104. Hence, the operator can perform the operations of, for example, powering on the apparatus, transferring the list of patients for imaging, referring to the list, of patients for imaging, processing a captured image, transferring image information, and powering off the apparatus while keeping the X-ray generation unit 101 stored.

Note that if the monitor 104 is partially covered by the X-ray generation unit 101 in the state in which the X-ray generation unit 101 is stored, the monitor 104 may be controlled to display an operation function or information on an uncovered portion of the monitor 104. More specifically, for example, when the computer has determined that the X-ray generation unit. 101 is arranged at the predetermined storage location, the display of the monitor 104 is changed based on it. Hence, an operation or information reference is possible even if the X-ray generation unit 101 partially blocks the display of the monitor 104 because of its physical structure.

### Second Embodiment

In the first embodiment, an example has been described, with reference to FIG. 3 in which the stored X-ray generation unit 101 and the monitor 104 are spaced apart in the horizontal, direction by a predetermined distance or more. However, the present invention is not limited to this. In the following embodiment, another detailed example will be explained.

FIG. 4 illustrates a portable X-ray diagnostic apparatus in which the position of a stored X-ray generation unit 101 in the vertical direction and the position of a monitor 104 in the vertical direction are spaced apart by a predetermined distance or more. As a result, when the X-ray generation unit 101 is stored, the monitor 104 is not covered by the X-ray generation unit 101 when viewed from the rear side at a predetermined angle (45° in FIG. 4) in the traveling direction of the cart. The predetermined distance is set such that the angle made by the horizontal plane and a line that connects a predetermined point (for example, a point on the front side of the display area) on the boundary of the monitor 104 and a point (for example, the hindmost point on the boundary of the X-ray generation unit 101) on the boundary of the X-ray generation unit 101 becomes a predetermined angle or more.

Note that the predetermined angle may be decided in accordance with, for example, the body height of the operator. To obtain the visibility of the monitor 104 from the predetermined angle, the storage location of the X-ray generation unit 101 may be set in the vertical direction or horizontal direction. That is, the portable X-ray diagnostic apparatus may have a function of obtaining the operator's body height via, for example, the monitor 104, and control the storage location of the X-ray generation unit 101 in accordance with the body height and the arrangement height of the monitor 104.

For example, the larger the operator's body height is, the larger the predetermined angle may be set. In this case, the storage location of the X-ray generation unit in the vertical direction and the position of the monitor 104 in the vertical direction may be spaced apart by a longer distance. In a similar case, the storage location of the X-ray generation unit in the horizontal direction and the position of the monitor 104 in the horizontal direction may be spaced apart by a longer distance. Note that the storage location of the X-ray generation unit in the horizontal direction is supposedly decided by an extendable boom 102. In this case, for example, the monitor 104 may be moved in the horizontal direction to ensure the distance in the horizontal direction. A high operability can thus be implemented even when the operator is tall.

If the operator is short, the predetermined angle may be small. The storage location of the X-ray generation unit in the vertical direction and the position of the monitor 104 in the vertical, direction may be made closer. Similarly, the storage location of the X-ray generation unit in the horizontal direction and she horizontal position of the monitor 104 may be made closer. This facilitates movement of the portable X-ray diagnostic apparatus when X-ray imaging is not performed.

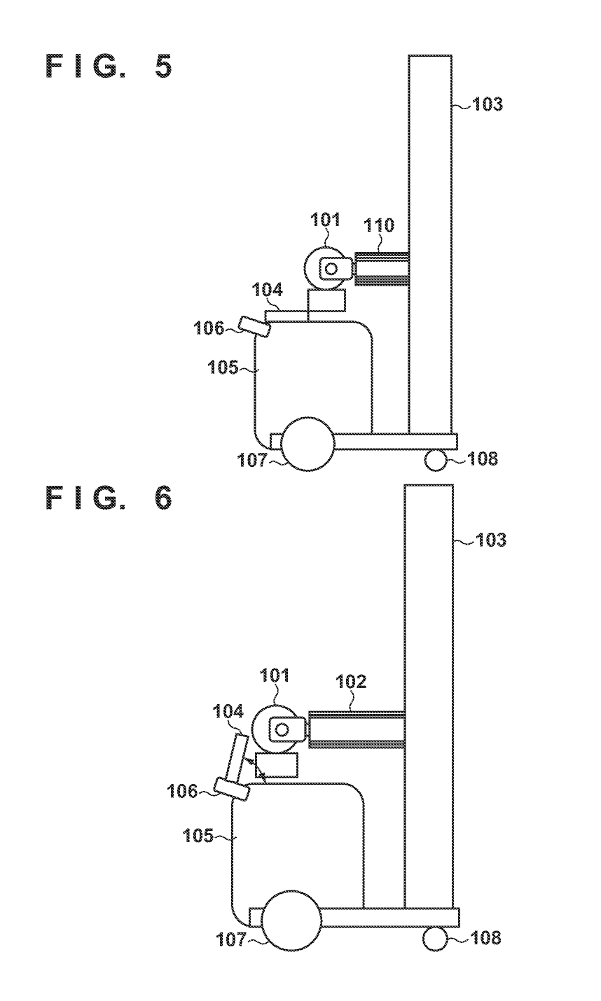

Note that the nested structure of the extendable boom 102 may be formed as a multistage structure including four or more stages, as shown in FIG. 5, so that the storage location of the X-ray generation unit 101 in the horizontal direction and the horizontal position of the monitor 104 are spaced part by a distance longer than in a case in which an boom of three or less staves is used. That is, the extendable be may include a first boom supported by a column 103 and second booms of three or more stages connected to the first boom. Out of the second booms of three or more stages, the second boom corresponding to the end of the extendable boom 102 supports the X-ray generation unit 101. The second booms of three or more stages may have the same movable range. Note that the “same movable range” here indicates that the second booms have almost equal movable ranges with a difference within a predetermined range (for example, several cm). That is, the movable ranges need not completely match. When the second booms of three or more stages are used, the minimum length of the whole boom can be reduced while ensuring a wide movable range. It is therefore possible to move the portable X-ray diagnostic apparatus more compactly. In, for example, a telescopic boom, the second booms of three or more stages are made to have the same movable range, thereby efficiently arranging the X-ray generation unit 101 in a wide range. The length of the boom at the storage location is thus decided such that the contents of information displayed on the monitor 104 are visible when the X-ray generation unit 101 is stored. This makes it possible to execute access and operations on the information displayed on the monitor 104 when moving the portable X-ray diagnostic apparatus.

As disclosed in Japanese Patent. No. 4515921, when the extendable boom 102 is joined to the side surface or upper portion of the column 103 in the expanding direction, the length of the boom when storing the X-ray generation unit 101 can be suppressed. Hence, when the extendable boom 102 is joined to the side surface or upper portion of the column 103 in the expanding direction, the position of the X-ray generation unit 101 in the horizontal direction and the horizontal position of the monitor 104 when storing the X-ray generation unit can sufficiently be spaced apart even if the space of the cart is small.

### Third Embodiment

In this embodiment, the storage location of an X-ray generation unit 101 is set on the rear surface of a monitor 104, as shown in FIG. 6. FIG. 6 illustrate an example in which the set angle of the monitor 104 is changed, and the X-ray generation unit 101 is stored on a side opposite to the surface to perform display and operations, that is, on the rear surface of the monitor 104. As for the set angle at this time, for example, the angle made by the horizontal plane and the surface of the monitor to perform display and operations is set to be a predetermined angle or more. The monitor 104 may have the set angle as shown in FIG. 6 as the basic position, or move when storing the X-ray generation unit 101. The monitor 104 may be moved interlockingly with the storage of the X-ray generation unit 101. For example, the portable X-ray diagnostic apparatus may control the monitor 104, an extendable boom 102, and a column 103 so that the form shown in FIG. 6 is obtained automatically upon detecting depression of a button to instruct the end of X-ray imaging on the monitor 104.

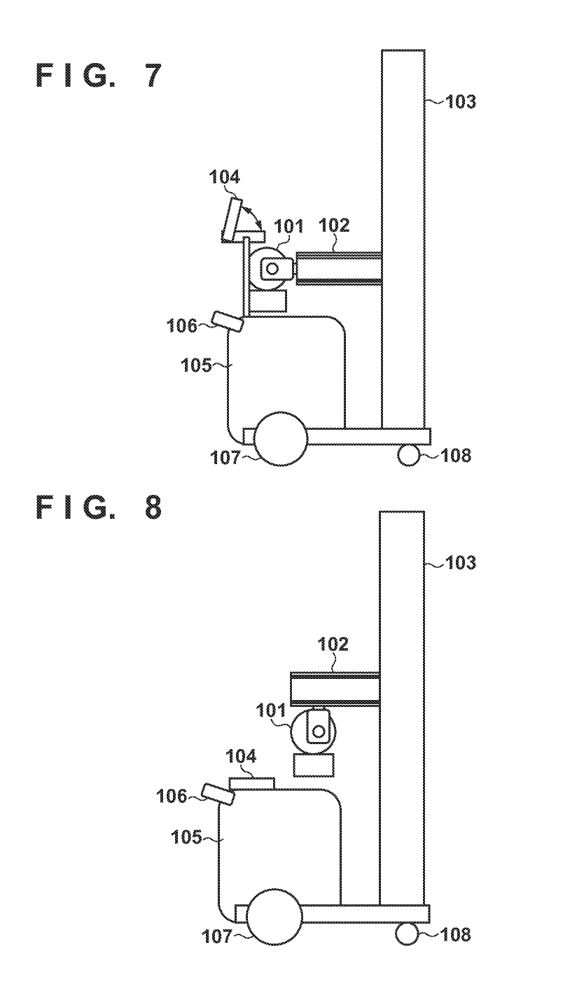

Note that not the set angle but the position of the monitor 104 may be moved. For example, as shown in FIG. 7, the set position of the monitor 104 may be moved to the upper side of the storage location of the X-ray generation unit 101. The set position of the monitor 104 may permanently be located above the storage location of the X-ray generation unit 101, as shown in FIG. 7. In this case as well, the set angle of the monitor 104 may be changeable. For example, when storing the X-ray generation unit 101, the monitor 104 may be raised by 90° from the horizontal direction not to collide against the X-ray generation unit 101. When the X-ray generation unit 101 has reached the storage location, the angle may be returned to the initial state. Alternatively, a predetermined angle when storing the X-ray generation unit 101 may be set in advance, the set angle of the monitor 104 may be controlled to that angle when the X-ray generation unit 101 has reached the storage location.

### Fourth Embodiment

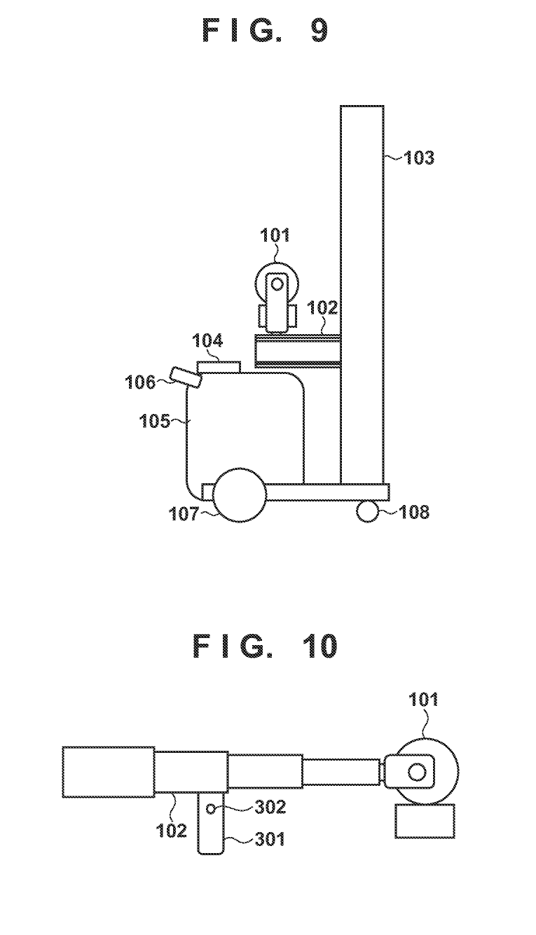

In the above-described first to third embodiments, a case has been described in which the relative positions of the X-ray generation unit 101 and the extendable boom 102 are fixed. In this embodiment, an example will be explained in which an X-ray generation unit 101 and an extendable boom 102 are movably joined. FIG. 8 is a view schematically showing a portable X-ray diagnostic apparatus that moves the X-ray generation unit 101 to the lower side of the extendable boom 102. The total length of the X-ray generation unit 101 and the extendable boom 102 in the horizontal, direction decreases when the X-ray generation unit 101 is moved to the lower side of the extendable boom 102. Hence, the storage location of the X-ray generation unit 101 in the horizontal direction and the horizontal position of a monitor 104 can easily be spaced apart by a predetermined distance or more. The same effect can be obtained even when the X-ray generation unit 101 is moved to the upper side of the extendable boom 102, as shown in FIG. 9.

Note that the above-described methods of storing the X-ray generation unit 101 can appropriately be combined. That is, in addition to spacing the X-ray generation unit 101 and the monitor 104 apart by a predetermined distance or more in the horizontal direction, as in this embodiment, they may be spaced apart by a predetermined distance or more in the vertical direction as well. These methods are combined, thereby improving the operability of the portable. X-ray diagnostic apparatus even when the space on the cart is small.

### Fifth Embodiment

In the above-described embodiments, a portable X-ray diagnostic apparatus including the extendable boom 102 has been described. In this portable X-ray diagnostic apparatus, the operator moves the portable X-ray diagnostic apparatus to one side of the patient's bed, releases the lock mechanism of the support mechanism of the X-ray generation unit 101, rotates the X-ray generation unit 101 to the bed side, moves to the opposite side of the bed, and aligns the X-ray tube. However, it may be not easy to go around to the opposite side of the patient's bed due to the environment of the hospital room or the like. For example, as for X-ray imaging in an operation room, the doctor is performing a surgical operation on the opposite side of the bed, and the operator cannot go around. In addition, adjusting the position of the X-ray generation unit 101 above the surgical field may damage the purity of the surgical field. If the operator cannot go around to the opposite side of the bed, the position adjustment of the portable X-ray diagnostic apparatus with the extendable boom 102 is difficult, resulting in poor operability.

In this embodiment, to facilitate position adjustment of an X-ray generation unit 101 even when the operator cannot go around to the opposite side of the bed, a multistage boom is used as an extendable boom 102. A handle and a lock release button of the support mechanism are provided on the intermediate stage of the boom. When the operator expands or contracts the intermediate stage of the boom with the handle using the handle, the expansion or contraction operation of the other stages is performed interlockingly. This allows the operator to perform the expansion or contraction operation of the extendable boom 102 near a column 103, he/she need not go around to the opposite side of the bed. Additionally, the operator need not perform the operation immediately above the surgical field during a surgical operation or the like, the purity can be ensured.

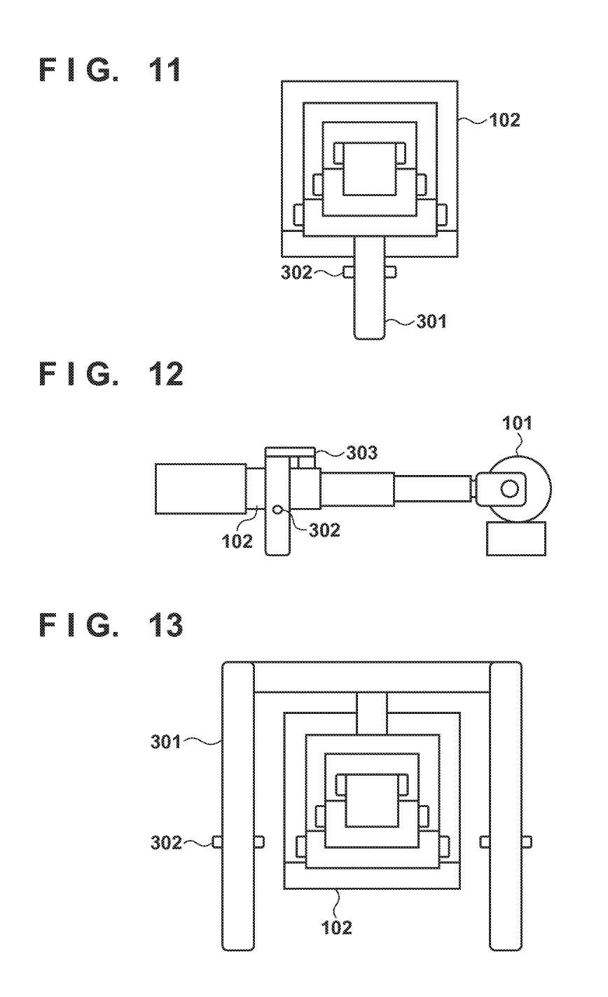

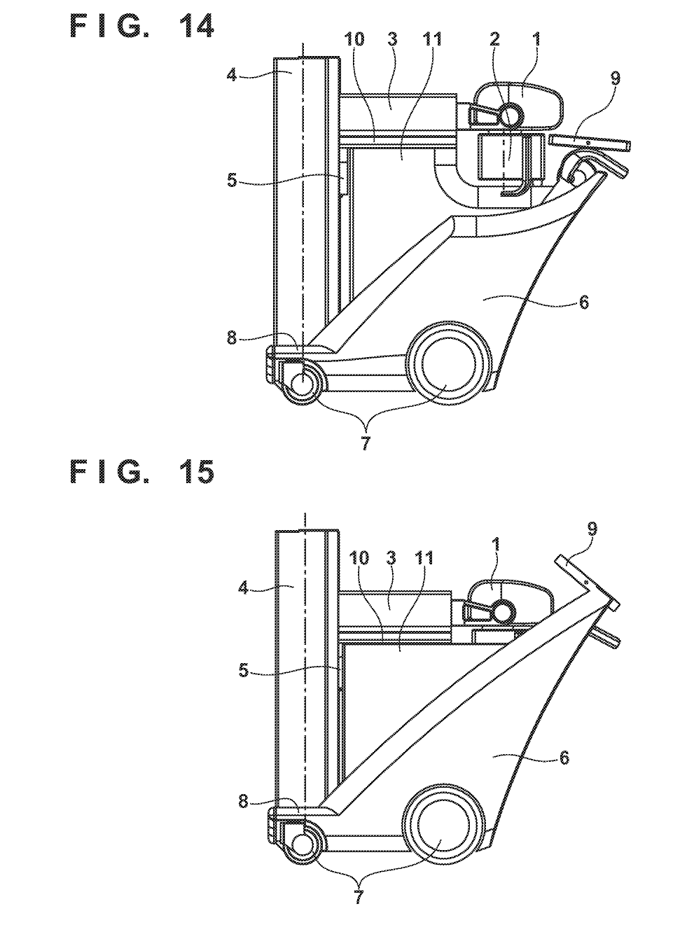

More specifically, the extendable boom is constituted as shown in FIGS. 10 to 13. FIG. 10 is a side view of a multistage extendable boom including a handle set on the lower side of the second stage. FIG. 11 is a sectional view of the multistage extendable boom in this case. Similarly, FIG. 12 is a side view of a multistage extendable boom including a handle set on the upper side of the second stage. FIG. 13 is a sectional view of the multistage extendable boom in this case.

In any case, a handle 301 is provided with a lock release button 302 of the support mechanism. The operator can adjust the direction, vertical position, and length of the boom can be adjusted after the lock is released by pressing the lock release button 302. Note that the “support mechanism” indicates the column 103 and the extendable boom 102. When they are locked, the operator can neither rotate the column nor change the vertical position of the extendable boom and adjust its length. Hence, the arrangement of this embodiment enables the operator to easily move the X-ray generation unit 101 to a predetermined position only by manipulating the handle 301 and the lock release button 302 set on it.

Note that in the above description, the handle is set on the upper or lower side of the second stage. However, even when the handle is set on a side surface or at another position, the same effect as described above can be obtained. In addition, the handle may be set not on the second stage but on, for example, the third stage of a four-stage extendable boom.

### Sixth Embodiment

In the first to fifth embodiments, an example has been described in which only the extendable boom 102 can expand or contract. In this embodiment, however, a case in which a column 103 is also extendable will be described. FIG. 14 illustrates a state in which an X-ray generation unit is placed in a storage portion formed as a concave portion to store the X-ray generation unit in a portable. X-ray diagnostic apparatus. When the storage portion is provided, the X-ray generation unit is hardly affected by external impact, and the portable X-ray diagnostic apparatus can be prevented from degrading.

Referring to FIG. 14, the X-ray diagnostic apparatus includes, for example, an X-ray tube 1, a collimator 2, an boom 3, a column 4, an boom support 5, a cart unit 6, a moving mechanism 7, a column rotation unit 8, a monitor 9, and a storage rod receiver 11. The X-ray generation unit 101 of the above-described embodiments includes, for example, the X-ray tube 1 and the collimator 2. The extendable boom 102 corresponds to the boom 3. The column 103 corresponds to the column 4. The monitor 104 corresponds to the monitor 9. The basic arrangement is the same as in the above described embodiments.

The X-ray tube 1 irradiates a target with X-rays. The collimator 2 restricts the X-ray irradiation range set on the X-ray tube 1. The boom 3 supports the X-ray tube 1, and has an expansion or contraction function of moving the X-ray tube 1 at least in the horizontal direction and an expansion or contraction position fixing function. The column 4 supports the boom 3. The boom support 5 connects the boom 3 and the column 4, and has a function of moving the boom 3 along the column 4 and a function of fixing the boom 3 at an arbitrary position. The cart unit 6 supports the column 4. The moving mechanism 7 can move the cart unit 6. The moving mechanism 7 rotates, for example, a plurality of tires or casters set on the ground, thereby moving the cart unit 6. The column rotation unit 8 connects the cart unit 6 and the column 4 and forms bearings, thereby making the column 4 rotatable on the cart unit 6 about a shaft perpendicular to the ground. The column rotation unit 8 also forms an off brake and can stop the rotation of the column 4 at an arbitrary position in the on state of the off brake. The monitor 9 is set at a position not to contact the stored tube on the bottom surface side of the storage portion to store the tube. The monitor 9 displays lists of information of patients scheduled for imaging upon round visits and the locations and test information of the patients. It is also possible to perform operations of setting imaging conditions and transmitting captured X-ray images to an in-hospital network. The storage rod receiver 11 includes a contact sensor that senses the contact or proximity of a boom lower surface 10.

In the example of FIG. 14, a concave portion to store the X-ray generation unit is formed in the cart unit 6. When X-ray imaging is not performed, the X-ray generation unit moves to the storage portion formed as the concave portion. At this time of storage, the storage rod receiver 11 and the boom lower surface 10 come into contact or approach to a predetermined distance or less. Note that in FIG. 14, the boom lower surface 10 need not have a convex portion projecting from the boom. A magnet and a magnetic sensor may be provided on the boom lower surface 10 and the storage rod receiver 11 provided on the cart unit 6, respectively, to sense that the boom 3 or the X-ray tube 1 is placed at the storage location. The apparatus as shown in FIG. 14 may be configured to permit only two behaviors, that is, vertical movement of the boom support 5 with respect to the column 4 and rotation of the column from brake release of the column rotation unit 8 when taking out the tube. The apparatus shown in FIG. 14 may permit only the behavior of vertical movement of the boom support 5 with respect to the column 4 when taking out the tube. The expansion or contraction of the boom 3 may be stopped by controlling the expansion or contraction position fixing unit of the boom 3. These can further prevent the X-ray tube 1 from coming into contact with the monitor 9.

When the column is also extendable, as shown FIG. 14, the size when the X-ray generation unit is stored becomes small. When a concave portion is provided, as shown in FIG. 14, and the X-ray generation unit is stored there, the size at the time of storage becomes smaller. This further facilitates movement of the X-ray diagnostic apparatus.

Note that although the X-ray tube 1 and the collimator 2 are exposed in the X-ray diagnostic apparatus shown in FIG. 14, side wall portions may be formed on the storage portion to partially cover them. FIG. 15 is a view showing an example of the X-ray diagnostic apparatus with such side wall portions provided on the storage portion. The X-ray tube 1 and the collimator 2 are partially covered. This can protect the X-ray tube 1 and the collimator 2 from external impact and prevent the X-ray diagnostic apparatus from degrading. Note that when a concave portion without side wall portions is formed to store the X-ray generation unit, as shown in FIG. 14, the operator can access the X-ray generation unit from a side with respect to the traveling direction of the X-ray diagnostic apparatus. Hence, for example, it is unnecessary to store the X-ray generation unit from above or first move the boom upward at the time of imaging, and restrictions on the boom operation decrease. In addition, maintenance can easily be done to, for example, exchange the X-ray tube at the time of storage.

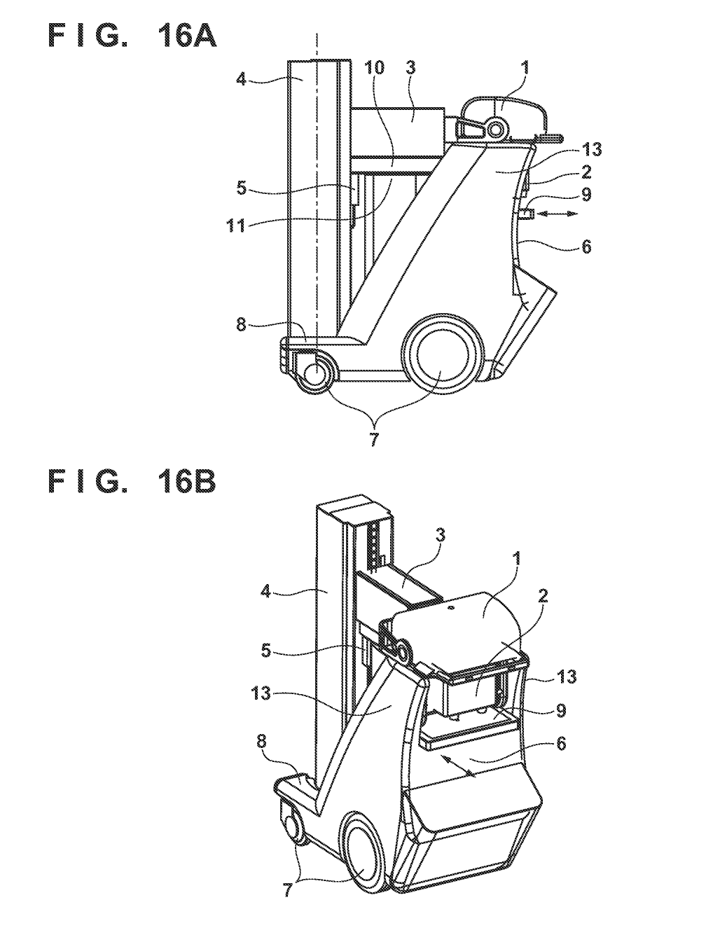

FIGS. 16A and 16B illustrate another example of the X-ray diagnostic apparatus with side wall portions provided on the storage portion. In this X-ray diagnostic apparatus, exposure protective walls projecting from the edges of the monitor 9 cover the stored X-ray tube. Referring to FIGS. 16A and 16B, the exposure protective walls have the same height as the storage rod receiver 11, and are so high as to cover at least the collimator 2. This reduces the possibility that the collimator 2 receives external impact at the time of storage, thus contributing to increase the life of the apparatus. The computer for control may have a table of rotation angles to prevent contact between the X-ray tube 1 and the exposure protective walls of the cart unit 6 and thus determine whether the X-ray tube 1 is coming close to the exposure protective walls of the cart unit 6. This can prevent the X-ray tube 1 from coming into contact with not only the monitor 9 but also the exposure protective walls of the cart unit 6.

Note that in the X-ray diagnostic apparatus shown in FIGS. 16A and 16B, the monitor 9 has its two opposing side surfaces slidably attached to monitor guide rails (not shown), and can be slid to put in and take out. For example, the monitor 9 is configured to be slidable in the directions of arrows in FIGS. 16A and 16B. Hence, during movement as well, the monitor 9 can be slid and taken out to obtain information or operate the X-ray diagnostic apparatus.

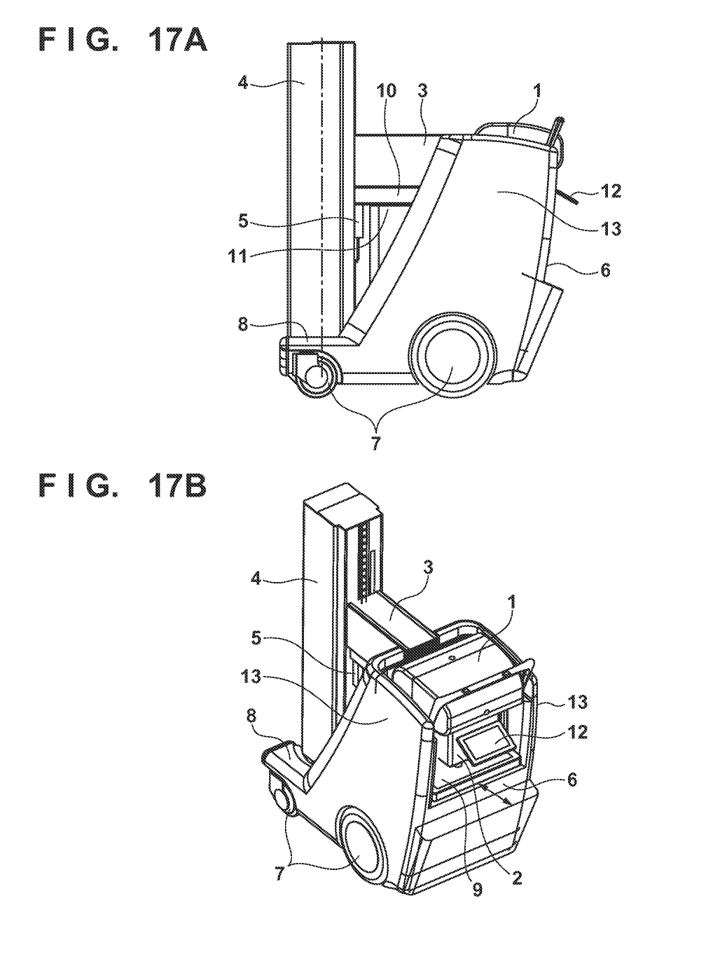

On the other hand, in an X-ray diagnostic apparatus shown in FIGS. 17A and 17B, two exposure protective walls 13 project from the cart unit 6 to cover the side surfaces of the collimator 2, and also extend up to a position to cover at least part of the X-ray tube 1. This further reduces the possibility that the collimator 2 and the X-ray tube 1 receive external impact at the time of storage, as compared to the case in FIGS. 16A and 16B, thus contributing to a long life of the apparatus. Note that the storage portion of the X-ray diagnostic apparatus shown in FIGS. 17A and 17B has a groove corresponding to the width of the boom 3 and fitted on the boom 3. When storing the X-ray generation unit, the boom 3 is first contracted and rotated in a direction reverse to the traveling direction. After that, the position (height) of the boom 3 in the direction of the column 4 is lowered, thereby storing the X-ray generation unit. If the groove is formed, the boom can be stored at a lower position. It is therefore possible to make the apparatus compacter and minimize the externally exposed portions of the X-ray tube 1 and the collimator 2. When the position of the boom at the time of storage of the X-ray generation unit lowers, front visibility improves to facilitate movement of the portable X-ray diagnostic apparatus.

Note that in the example shown in FIGS. 17A and 17B, the apparatus includes a second monitor 12 belonging to the collimator 2 in addition to the first monitor 9 arranged on the medical cart main body or the cart unit 6. The first monitor 9 is configured to be slidable in the directions of arrows in FIGS. 17A and 17B, as in the X-ray diagnostic apparatus shown in FIGS. 16A and 16B. The second monitor 12 constitutes a so-called vari-angle display unit. For example, the second monitor 12 is fixed to the collimator 2 along one side of the display screen. The orientation of the second monitor 12 can be changed using the one side as an axis. The method of fixing the second monitor 12 to the collimator 2 is not limited to this. For example, the second monitor 12 may be configured to be able to change its orientation within a predetermined angle range in an arbitrary direction with respect to the collimator 2. The second monitor 12 may be set on the surface portion of a unit to which the X-ray tube 1 at the time of storage is attached.

In the example shown in FIGS. 17A and 17B, when the X-ray tube 1 and the collimator 2 are stored, the second monitor 12 is powered on while powering off the first monitor 9 under the control of the computer for control so that the second monitor 12 can display information and accept operations. Instead of or in addition to this, the slidable first monitor 9 and the second monitor 12 can be caused to display different pieces of information to effectively use the display areas, thereby increasing the convenience of the operator.

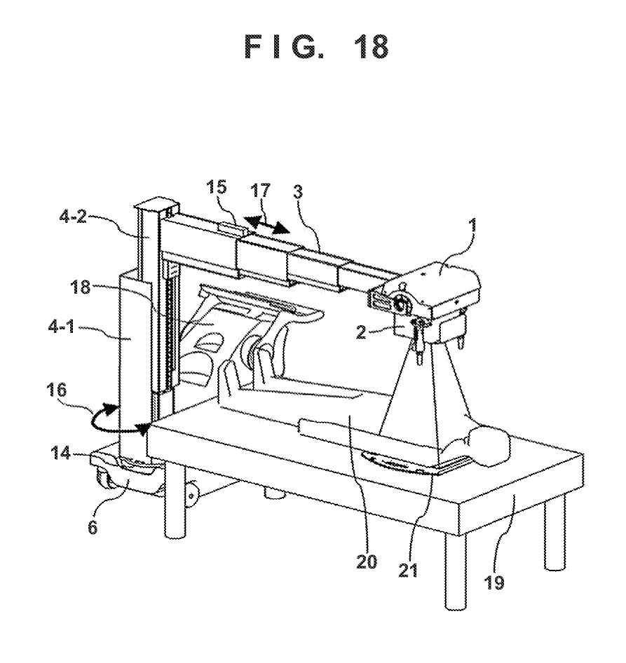

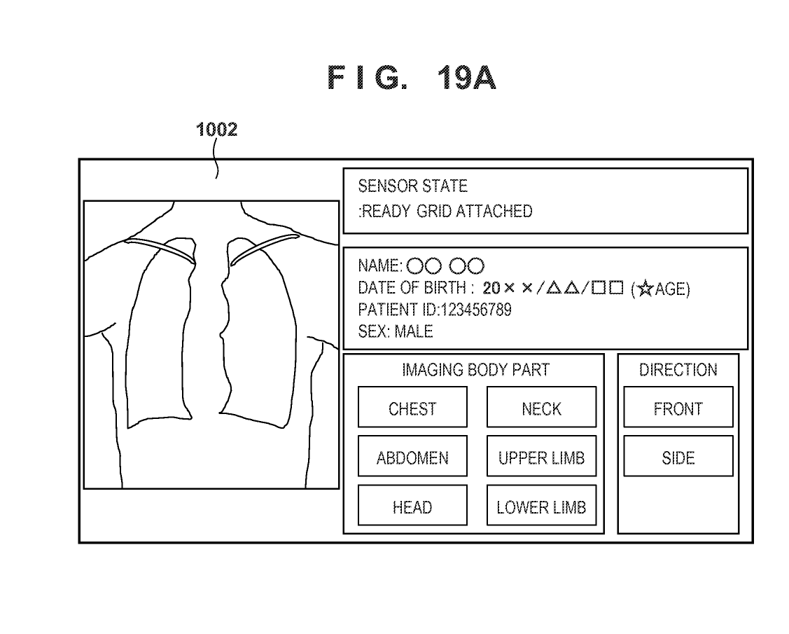

FIG. 18 illustrates a state in which when the column 4 can expand or contract in multistage, the boom 3 has a nested or telescopic structure, and each member can expand or contract in accordance with the operator's operation, the column and the boom are expanded, and X-ray imaging is performed. A system control, unit 18 including an X-ray high voltage generator, an X-ray controller, and a control panel is mounted on the cart unit 6. The system control unit 18 controls X-rays as well. A handle for movement and a display capable of displaying X-ray irradiation information and inputting an irradiation instruction are arranged on the system control unit 18. A first column 4-1 is set and arranged vertically above the front side of the cart unit 6 to be capable of omnidirectional turn 16 with respect to the cart unit 6. The turn amount is confirmed by a pivotal displacement sensor 14. A second column 4-2 capable of moving in the vertical direction (axial direction of the column) along the column is formed in the first column 4-1. If the apparatus is exclusively used in a normal hospital room, and a high position is not particularly needed, the first column 4-1 and the second column 4-2 may be integrated into a single column. The apparatus is also provided with the boom 3 that supports the X-ray tube and can contract in a horizontal direction 17 almost perpendicular to the second column 4-2. The moving amount is confirmed by a displacement sensor 15. A unit including the X-ray tube 1 is attached to the end of the boom 3. The collimator 2 is attached to the lower side of the X-ray tube 1. A subject 20 is lying on a bed 19 arranged in the hospital room, and a flat panel 21 for imaging is arranged between them. When each of the column and the boom has a multistage structure, the apparatus becomes compact at the time of storage, the front visibility can be ensured, and handling becomes easy.

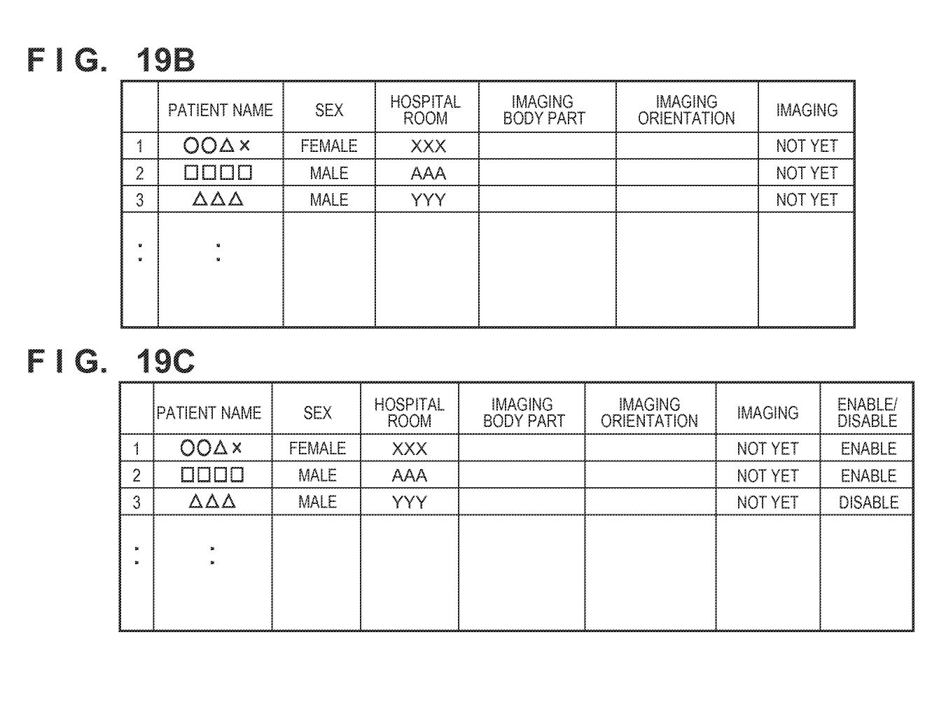

FIG. 19A is an explanatory view of display of the monitor 9. FIG. 19A shows a normal monitor display state. In this embodiment, a state in which the X-ray tube 1 is placed at the storage location (when moving the apparatus) is considered. Pieces of information shown in FIGS. 19A to 19C are displayed on the monitor 9 under the control of the computer for control included in the X-ray diagnostic apparatus.

In the normal state shown in FIG. 19A, the whole monitor display enable area is used as a display use area 1002. In FIG. 19A, for example, a radiographic image obtained by immediately preceding imaging is displayed. In another example, whether the X-ray sensor is ready for imaging or not and whether a scattered ray removing grid is attached or not are displayed. In addition, imaging information and patient information such as information (name, date of birth, age, patient ID, and sex) of the imaging target subject (patient), an imaging target body part or a list of selectable imaging body parts, and an imaging direction or a list of selectable imaging directions may be displayed.

FIGS. 19B and 19B are explanatory views of another display of the monitor 9. The monitor 9 can display, for example, a list to display subjects who have not undergone imaging yet or imaging requirements, as shown in FIG. 19B. Such a list is based on management information obtained via wireless connection from, for example, a RIS (Radiology information system). As shown in FIG. 19C, for example, the list shown in FIG. 19B may include imaging enable/disable information of each subject. The imaging enable/disable information is obtained by obtaining, from the RIS, information representing whether imaging has been canceled due to, for example, patient's circumstances.

According to the present invention, it is possible to improve the operability of the portable X-ray diagnostic apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application. No. 2012-218461 filed on Sep. 28, 2012, which is hereby incorporated by reference herein in its entirety.

What is claimed is:

1. A portable X-ray diagnostic apparatus comprising:
an X-ray generation unit configured to generate X-rays;
a display unit configured to display information about X-ray imaging by said X-ray generation unit;
a column including a shaft extending in a first direction and configured to be rotatable about the shaft;
a cart on which said display unit and said column are set; and
an extendable boom configured to support said X-ray generation unit and extend in a second direction crossing the first direction, said boom obtaining, when storing said X-ray generation unit, a length to store said X-ray generation unit between said display unit and said column.

2. The apparatus according to claim 1, wherein said boom supports said X-ray generation unit at an end of said boom, and changes a position of said X-ray generation unit in accordance with expansion or contraction of said boom.

3. The apparatus according to claim 1, wherein said boom includes a first boom supported by said column, and second booms of at least three stages movably connected to said first boom in the second direction, and
out of said second booms of at least three stages, a second boom located at an end of said boom supports said X-ray generation unit.

4. The apparatus according to claim 3, wherein said second booms of at least three stages have movable ranges substantially equal to each other.

5. The apparatus according to claim 1, wherein when said boom is contracted to a minimum length and arranged along a direction reverse to a traveling direction of the portable X-ray diagnostic apparatus, said X-ray generation unit is arranged on a front side of said display unit in the traveling direction, and said column is arranged on a front side of said X-ray generation unit in the traveling direction.

6. The apparatus according to claim 1, wherein when said X-ray generation unit is placed at a storage location when moving the portable X-ray diagnostic apparatus, the length of said boom at the storage location is decided to make information displayed on said display unit visible.

7. The apparatus according to claim 6, wherein the storage location of said X-ray generation unit is a position of said X-ray generation unit when said boom is contracted to a predetermined length and arranged along a direction reverse to a traveling direction of the portable X-ray diagnostic apparatus.

8. The apparatus according to claim 1, wherein said column includes a first column set to be rotatable with respect to said cart, and a second column arranged to be movable with respect to said first column in a direction of an axis of said column, and
said boom is supported by said second column.

9. The apparatus according to claim 8, wherein said boom is arranged to be movable with respect to said second column in the direction of the axis of said column.

10. The apparatus according to claim 1, wherein a storage portion to store said X-ray generation unit at a storage location when the portable X-ray diagnostic apparatus moves is formed in said cart.

11. The apparatus according to claim 10, wherein said storage portion includes a concave portion formed in said cart.

12. The apparatus according to claim 10, wherein said X-ray generation unit comprises a collimator, and
said storage portion comprises a side wall portion configured to cover at least a side surface of said collimator.

13. The apparatus according to claim 10, wherein a groove to be fitted on said boom at the storage location is formed in said storage portion in correspondence with a width of said boom.

14. The apparatus according to claim 10, wherein said X-ray generation unit comprises a collimator, and
said display unit is arranged above said collimator when said X-ray generation unit is stored in said storage portion.

15. A portable X-ray diagnostic apparatus comprising an X-ray generation unit configured to generate X-rays, a support mechanism configured to support said X-ray generation unit and move said X-ray generation unit to a predetermined position, and an operation display unit configured to display information about X-ray imaging and accept an operation,
wherein a storage location of said X-ray generation unit when the X-ray imaging is not performed is decided such that the storage location and a position of said operation display unit are spaced apart in at least one of a horizontal direction and a vertical direction by not less than a predetermined distance.

16. The apparatus according to claim 15, wherein the predetermined distance is set such that an angle made by a horizontal plane and a line that connects one point on a boundary of said X-ray generation unit and one point on a boundary of said operation display unit becomes not less than a predetermined angle.

17. The apparatus according to claim 15, further comprising an obtaining unit configured to obtain a body height of an operator of the portable X-ray diagnostic apparatus,
wherein the predetermined distance is decided in accordance with the body height of the operator.

18. A portable X-ray diagnostic apparatus comprising an X-ray generation unit configured to generate X-rays, a support mechanism configured to support said X-ray generation unit and move said X-ray generation unit to a predetermined position, and an operation display unit configured to display information about X-ray imaging and accept an operation,
wherein said X-ray generation unit when the X-ray imaging is not performed is stored on a rear surface of a surface of said operation display unit to perform display and an operation.

19. The apparatus according to claim 18, wherein an angle made by a horizontal plane and the surface of said operation display unit to perform display and the operation is not less than a predetermined angle.

20. The apparatus according to claim 18, wherein said operation display unit is located above said X-ray generation unit when the X-ray imaging is not performed.

21. The apparatus according to claim 18, wherein when storing said X-ray generation unit, said operation display unit moves.

22. The apparatus according to claim 15, further comprising a determination unit configured to determine whether said X-ray generation unit is stored,
wherein upon determining that said X-ray generation unit is stored, display of said operation display unit is changed.

23. The apparatus according to claim 15, wherein said support mechanism comprises a multistage extendable boom.

24. The apparatus according to claim 15, wherein said support mechanism comprises a column, and an extendable boom joined to said column and configured to expand or contract in a direction perpendicular to said column, and
said extendable boom is joined to one of a side surface and an upper portion of said column with respect to an expansion direction of said extendable boom.

25. The apparatus according to claim 15, wherein said support mechanism comprises a column, and an extendable boom joined to said column and configured to expand or contract in a direction perpendicular to said column,
said X-ray generation unit is movably joined to said extendable boom, and
when storing said X-ray generation unit, said X-ray generation unit is moved to one of an upper side and a lower side of said extendable boom.

26. The apparatus according to claim 15, wherein said support mechanism comprises a multistage extendable boom, and
a handle configured to operate said support mechanism is provided on one of intermediate stages of said multistage extendable boom.

27. The apparatus according to claim 26, wherein said handle includes a button to release lock of said support mechanism when moving said X-ray generation unit.

28. The apparatus according to claim 26, wherein when an expansion or contraction operation of said multistage extendable boom is performed on the intermediate stage to which said handle is attached, the expansion or contraction operation of other stages of said multistage extendable boom is also interlockingly performed.
